(19) United States
(12) Patent Application Publication
(10) Pub. No.: US 2021/0401493 A1
(43) Pub. Date: Dec. 30, 2021

(54) SPLIT BIPHASIC WAVEFORM FOR EMBOLIC REDUCTION

(71) Applicant: Medtronic, Inc., Minneapolis, MN (US)

(72) Inventors: Brian T. Howard, Minneapolis, MN (US); Mark T. Stewart, Lino Lakes, MN (US); Lars M. Mattison, St. Anthony, MN (US)

(21) Appl. No.: 17/357,272

(22) Filed: Jun. 24, 2021

Related U.S. Application Data
(60) Provisional application No. 63/043,406, filed on Jun. 24, 2020.

(51) Int. Cl. A61B 18/14

(52) U.S. Cl. CPC A61B 18/1492; A61B 2018/00577

(57) ABSTRACT

A method of ablating tissue with pulse field ablation energy includes generating a single pulse of energy between a first set of one or more conducting elements of a first polarity and a second set of one or more conducting elements of a second polarity, the single pulse of energy having a first pulse width and consecutively generating pulses of energy with opposite polarity to that of the single pulse of energy, the pulses having a collective pulse width equal to the first pulse width.

### CROSS-REFERENCE TO RELATED APPLICATION

This application claims the benefit of U.S. Application Ser. No. 63/043,406, filed Jun. 24, 2020.

### FIELD

The present technology is related to methods and apparatus for delivering pulse field ablation pulses.

### BACKGROUND

Pulsed field ablation (PFA) is a non-thermal energy delivery modality in which high voltage electric pulse fields are delivered to a target tissue region with the effect of hyperpermeablizing the cell membranes of the targeted tissues. Such exposure can result in stunning or killing these cells due to cell membrane destabilization.

As voltage is applied between anode and cathode elements in a conductive medium, such as blood, electrolysis is an expected result given certain conditions of time and duration. The electrolysis creates bubbles in aqueous fluids in body, such as blood or in tissue, the volume of which may be undesirable and diminish the effect of a PFA treatment. Further, the volume of gas formed by such a reaction can vary between the anodic and cathodic elements. This disparity between gaseous formation at opposing elements is paired with biphasic energy delivery. Biphasic deliveries may be generally understood as switching the polarity between conducting elements during a single waveform, such that for a first portion of the energy delivery one element may be the anode with a second element the cathode, and then for a second portion of the same energy delivery, the first element will serve as the cathode, and the second element as the anode. In this manner, current flows from the first element to the second element and then in reverse. This effect can be beneficial for aspects of cardiac ablation because driving current in both directions reduces the net charge imparted to the fluid/tissue/target, thereby making other measurements possible and more precise. Such measurements include resolving global and local electric activity of the cardiac tissue (ECG/EGMs), positions of catheters, impedance, temperature, etc.

### SUMMARY

The techniques of this disclosure generally relate to apparatus and methods for delivering high voltage pulse field pulses.

In general terms one or more first elements are located on or near the target tissue. A second set of one or more elements are located within or exposed to blood or fluid for which bubble formation poses a risk or other technical challenge. In a non-limiting example for clarity in blood, targeting cardiac tissue, and considering the first and second elements as singular with both phases of a biphasic delivery equal in magnitude; when the targeting first element is engaged as a cathode in opposition to the second element as an anode, a single long pulse width is delivered between the elements. Longer pulse widths are determined in many regimes to be more efficacious at ablating the cardiac tissue targeted but they will also cause more bubble formation at the cathodic element. Because the first element is targeting the tissue, it may be partially obscured from the blood pool by the contact or proximity, while the anodic second element with more relative exposure to the blood does not create as many bubbles during this portion of the therapy. Then during the second phase, when the polarity is reversed and the cathode is the second element with substantial exposure to the blood, shorter pulses are delivered which are less capable of producing bubbles on the cathodic element. The individually shorter, but more numerous, pulses during this phase may be less effective at cardiac ablation but will still have an effect (especially if preceding the single opposing phase pulse as preparatory for the tissue), and the main advantage being the maintenance of the biphasic waveform which has many advantages for cardia ablation in particular.

In one aspect, a method of ablating tissue with pulse field ablation energy includes delivering a single pulse of energy between a first set of one or more conducting elements of a first polarity and a second set of one or more conducting elements of a second polarity, the single pulse of energy having a first pulse width and consecutively delivering a plurality of pulses of energy with opposite polarity, the plurality of pulses having a collective pulse width substantially equal to the first pulse width.

In another aspect of this embodiment, the single pulse has a voltage between 300V and 4000V.

In another aspect of this embodiment, the plurality of pulses has a voltage between 300V and 4000V.

In another aspect of this embodiment, the tissue being ablated is cardiac tissue.

In another aspect of this embodiment, the first polarity and the second polarity are continually switched during subsequent deliveries of the single pulse of energy and the plurality of pulses of energy.

In another aspect of this embodiment, the delivering of the single pulse of energy and the delivery of the plurality of pulses of energy occurs between a first electrode of the first set of the one or more conducting elements and a second electrode of the second set of the one or more conducting elements.

In another aspect of this embodiment, the first set of one or more conducting elements is on a first medical device and the second set of one more conducting elements is on a second medical device different than the first medical device.

In another aspect of this embodiment, the delivering of the plurality of pulses of energy and the delivery of the single pulse of energy occurs from a first electrode and a second electrode.

In another aspect of this embodiment, the second electrode is larger than the first electrode.

In one aspect, a pulse field ablation energy generator includes processing circuitry being configured to deliver a single pulse of energy between a first set of one or more conducting elements of a first polarity and a second set of one or more conducting elements of a second polarity, the single pulse of energy having a first pulse width and consecutively deliver a plurality of pulses of energy with opposite polarity, the plurality of pulses having a collective pulse width substantially equal to the first pulse width.

In another aspect of this embodiment, the single pulse has a voltage between 300V and 4000V.

In another aspect of this embodiment, the plurality of pulses has a voltage between 300V and 4000V.

In another aspect of this embodiment, the processing circuitry is further configured to continually switch the first polarity and the second polarity during subsequent deliveries of the single pulse of energy and the plurality of pulses of energy.

In another aspect of this embodiment, the processing circuitry is configured to be in communication with a medical device, and wherein the delivering of the single pulse of energy and the delivery of the plurality of pulses of energy occurs from a first electrode of the first set of the one more conductive elements and a second electrode of the second set of one more conducting elements of the medical device.

In another aspect of this embodiment, the second electrode is larger than the first electrode.

In another aspect of this embodiment, the processing circuitry is configured to be in communication with a medical device, and wherein the delivering of the plurality of pulses of energy and the delivery of the single pulse of energy occurs from the first electrode and the second electrode of the medical device.

In another aspect of this embodiment the second electrode is larger than the first electrode.

In one aspect, a medical system includes a pulse field ablation energy generator including processing circuitry being configured to deliver a single pulse of energy between a first set of one or more conducting elements of a first polarity and a second set of one or more conducting elements of a second polarity, the single pulse of energy having a first pulse width; and consecutively deliver a plurality of pulses of energy with opposite polarity, the plurality of pulses having a collective pulse width substantially equal to the first pulse width. A medical device is included having a plurality of electrodes in communication with the generator, the medical device having a first tip electrode and a proximal second electrode, the first tip electrode and the second electrode being configured to deliver the plurality of pulses of energy and the single pulse of energy.

In another aspect of this embodiment, the processing circuitry is further configured to continually switch the first polarity and the second polarity during subsequent deliveries of the single pulse of energy and the plurality of pulses of energy.

In another aspect of this embodiment, both the single pulse of energy and the plurality of pulses of energy have a voltage of between 300V to 4000V.

In one aspect, a method of applying therapeutic electric fields includes delivering a first pulse of energy between a first and second set of electrodes with a first and second polarity respectively. A plurality of pulses of energy shorter individual duration, proximate in time to the first pulse of energy is delivered. The plurality of pulses has a collective pulse width substantially equal to the first pulse width. The first polarity is anodic relative to the second polarity during the delivery of the first pulse. The first polarity is cathodic relative to the second polarity during the delivery of the plurality of shorter pulses.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an assembly view of an exemplary pulse field ablation medical system constructed in accordance with the principles of the present application;

FIG. 2 is an exemplary pulse field ablation waveform for reducing bubble formation;

FIG. 3 is side view of an exemplary medical device configured to deliver the waveform shown in FIG. 2;

FIG. 4 is an exemplary pulse field ablation waveform for reducing bubble formation;

FIG. 5 is an exemplary two medical device configuration for delivering pulse field ablation;

FIG. 6 is a flow chart of an exemplary method of generating pulse field ablation pulses;

FIG. 7 is a flow chart of an exemplary method of generating pulse field ablation pulses; and

FIG. 8 is a flow chart of an exemplary method of generating pulse field ablation.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

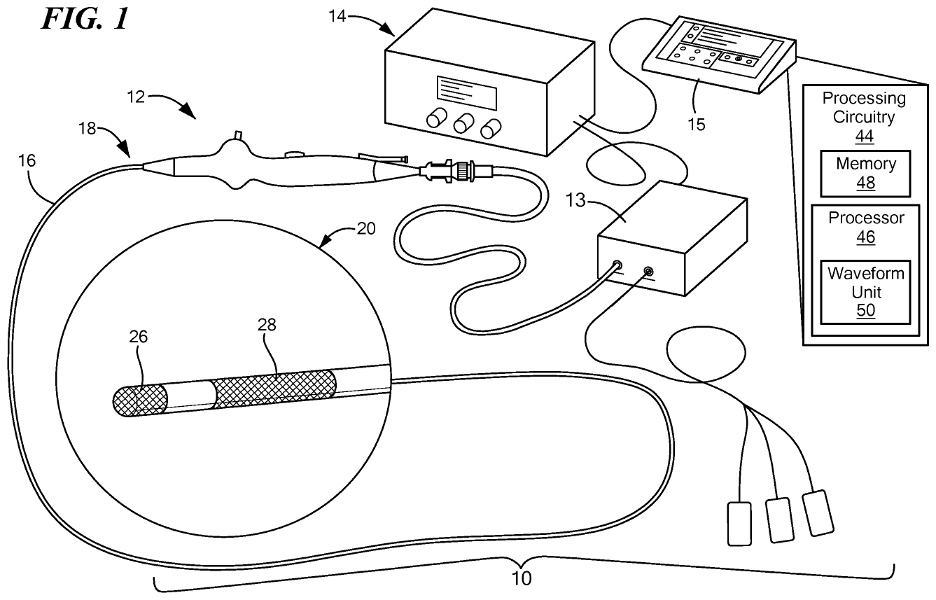

Referring now to the drawing figures in which like reference designations refer to like elements, an embodiment of a medical system constructed in accordance with principles of the present invention is shown in FIG. 1 and generally designated as “10.” The system 10 generally includes a medical device 12 that may be coupled directly to an energy supply, for example, a pulse field ablation generator 14 configured to generate and deliver various modalities of energy described herein. The pulse field ablation generator 14 may further be coupled directly or indirectly to a catheter electrode distribution system 13 configured to deliver the generated energy to the medical device 12. A remote controller 15 may further be included in communication with the generator 14, the controller 15 includes processing circuitry 44 configured to operate and control the various functions of the generator 14. Alternatively, the controller 15 may be integrated within the generator 14. The medical device 12 may generally include one or more diagnostic or treatment regions for energetic, therapeutic and/or investigatory interaction between the medical device 12 and a treatment site. The treatment region(s) may deliver, for example, pulsed electroporation energy or radiofrequency energy to a tissue area in proximity to the treatment region(s).

In one or more embodiments, the processing circuitry 44 may include a processor 46 and a memory 48. In particular, in addition to or instead of a processor, such as a central processing unit, and memory, the processing circuitry 44 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 46 may be configured to access (e.g., write to and/or read from) the memory 48, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

The processing circuitry 44 may be configured to control any of the methods and/or processes described herein and/or to cause such methods, and/or processes to be performed, e.g., by the remote controller 15. Processor 46 corresponds to one or more processors 46 for performing functions described herein. The memory 48 is configured to store data, programmatic software code and/or other information described herein. In some embodiments, the software may include instructions that, when executed by the processor 48 and/or processing circuitry 44 causes the processor 46 and/or processing circuitry 44 to perform the processes described herein with respect to remote controller 15. For example, processing circuitry 44 of the remote controller 15 may include waveform unit 50 that is configured to perform one or more functions described herein such as with respect to pulse generation and control.

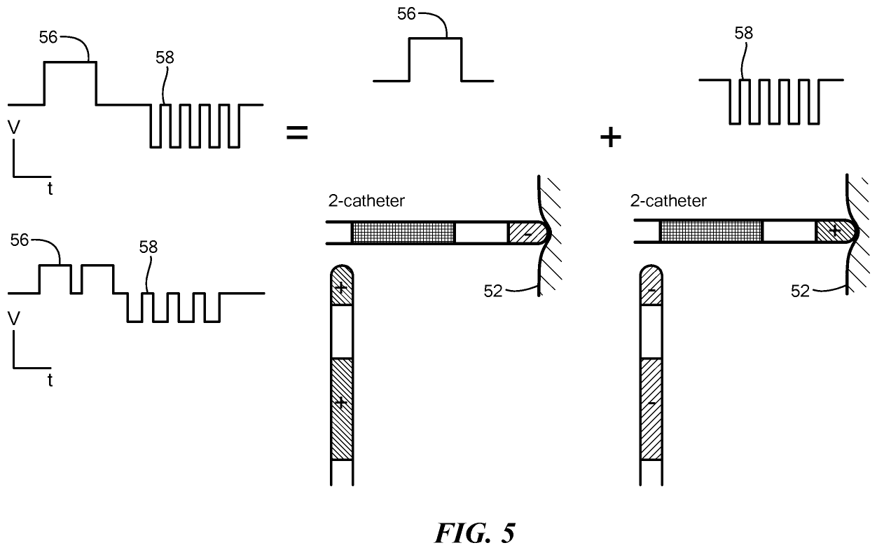

The medical device 12 may include an elongate body or catheter 16 passable through a patient's vasculature and/or positionable proximate to a tissue region for diagnosis or treatment, such as a catheter, sheath, or intravascular introducer. The elongate body or catheter 16 may define a proximal portion 18 and a distal portion 20, and may further include one or more lumens disposed within the elongate body 16 thereby providing mechanical, electrical, and/or fluid communication between the proximal portion of the elongate body 16 and the distal portion of the elongate body 16. The distal portion 20 may generally define the one or more treatment region(s) of the medical device 12 that are operable to monitor, diagnose, and/or treat a portion of a patient. The treatment region(s) may have a variety of configurations to facilitate such operation. In the case of purely bipolar pulsed field delivery, distal portion 20 includes electrodes 26 and 28 that form a bipolar configuration for energy delivery. In an alternate configuration, a plurality of the electrodes may serve as one pole while a second device containing one or more electrodes (see FIG. 5) would be placed to serve as the opposing pole of the bipolar configuration. For example, as shown in FIG. 1, the medical device 12 may be have a linear configuration with electrodes 26 and 28. In other configurations the medical device 12 may be configured as a focal catheter or an extended array that is transitionable from a linear to an arcuate or circular configuration. While the configuration of FIG. 1 illustrates two electrodes 26 and 28 more electrodes can be provided to deliver pulsed field energy.

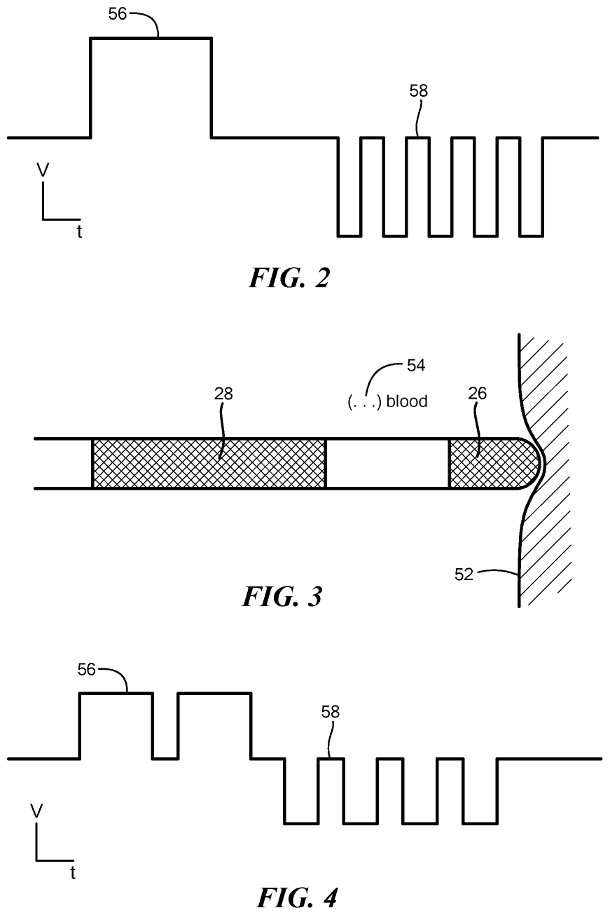

Referring now to FIGS. 2-6, in an exemplary configuration, a method of treating tissue, for example, cardiac tissue, includes generating and delivering a high voltage energy between 300V to 4000V to the target tissue region. The high voltage energy generated and delivered may have a therapeutic effect on the tissue. For example, the therapeutic effect may include stimulating tissue without causing damage, causing reversible electroporation, or irreversible electroporation. In one configuration a single pulse of energy 56 having a pulse width between 2 and 1000 μs and a voltage between 300V-4000V is generated and delivered from one of the electrodes 26 or 28 and a plurality of generally shorter pulses 58 with a pulse width generally between 0.5 to 100 μs and having a collective pulse width substantially equal or equal to the pulse width of the single pulse of energy and a voltage between 300V-4000V is delivered consecutively. Such a configuration may reduce bubble formation while still providing effective treatment.

For example, referring to FIG. 3, wherein a distal portion 20 is shown within a fluid (blood) environment 54 and pressed against tissue 52, the electrodes may include a first electrode 26, which may be a ring or a tip electrode, and one or more proximal electrodes 28. In the configuration shown in FIG. 3, the second electrode 28 is larger than the electrode 26 and may have a larger resistance. In one configuration, the electrode 26 has a first polarity, for example, positive, and the one or more proximal electrodes having a second polarity different than the first polarity, for example, negative. The polarities of the electrodes 26 and 28 may be fixed or may be switched during consecutive cycles of energy delivery. For example, in one configuration, the generator 14 may be configured fix the polarities of the electrodes 26 and 28, for example, all positive, all negative, or alternating polarities, during generation of the single pulse of energy or the short pulses. In another configuration, the polarities of one or more of the electrodes 26, 28 may be switched, for example, alternated, between positive and negative in consecutive deliveries of the single pulse or the shorter pulses. In one configuration, the single pulse and the plurality of pulses have the same voltage, but in other configurations they may have different voltages.

For example, as shown in FIG. 3, the first electrode 26 operates as the cathode having a first polarity and the second electrode 28 operates as the anode having a second polarity different than the first polarity. In an exemplary, method of use, the medical device 12, is positioned adjacent the tissue 52 to be treated, for example, cardiac tissue. The first electrode 26 is pressed against the tissue to be treated, with the second electrode 28 being positioned within or adjacent to blood 54. The first electrode 26 and the second electrode 28 are configured to deliver a single pulse of energy 56 for predetermined period of time. The single duration pulse 56 can be more effective at ablating tissue such as cardiac tissue while the plurality of pulses 58 of the same or substantially the same total duration as the single pulse from the first electrode 26 and the second electrode 28 limit bubble formation while maintaining useful characteristics of biphasic delivery of energy such as charge balance or reduction of stimulation response. In particular, because the longer pulses of energy can produce more bubbles than the plurality of pulses of energy at the cathode, the position of the first electrode 26 away from the blood during this phase reduces total bubble formation as compared with a delivery of the longer pulse during both phases of the biphasic delivery for example. The delivery of a single pulse of energy and a plurality of pulses of energy having a collective pulse width equal or substantially equal to the single pulse of energy from the first electrode 26 and the second electrode 28, may be repeated at the same biphasic cycle or may be automatically switched during consecutive deliveries of energy.

Referring now to FIG. 4, in still other configurations, the plurality of electrodes 26, 28 are configured to deliver a first plurality of longer pulse width pulses 56 at a first voltage between 300V and 4000V. The pulses may have the same or different voltages. Thereafter, a second plurality of shorter pulses 58 may be delivered at the same or different voltage as the first plurality of pulses. The combined pulse width of the first plurality of pulses may be the same or substantially the same as the second plurality of pulses. It is further not required that the individual pulses in the second plurality of shorter pulses share the same duration nor evenly spaced in time whereas they may be in an exemplary configuration.

In other configurations, as shown in FIG. 5, the first electrode 26 may be on a first medical device and the second electrode 28 may be on a second medical device proximate the first medical device and the first electrode 26 and the second electrode 28 are configured to deliver biphasic pulses between them in the manner discussed above. The first and second medical devices may be the same or different configurations such as focal, linear, splined, or basket, engaging single electrodes as individual poles or a plurality of electrodes or a subset of a plurality of available electrodes or delivering pulses between a plurality of electrodes on a multi-electrode device and an extensible counter-electrode such as an electrode formed as a distal portion of a guide-wire that is extended beyond the multi-electrode device to a more remote location in the vascular system, such as deep within a pulmonary vein. In still other configurations, any number of electrodes 26, 28 may be configured to deliver biphasic pulses discussed above.

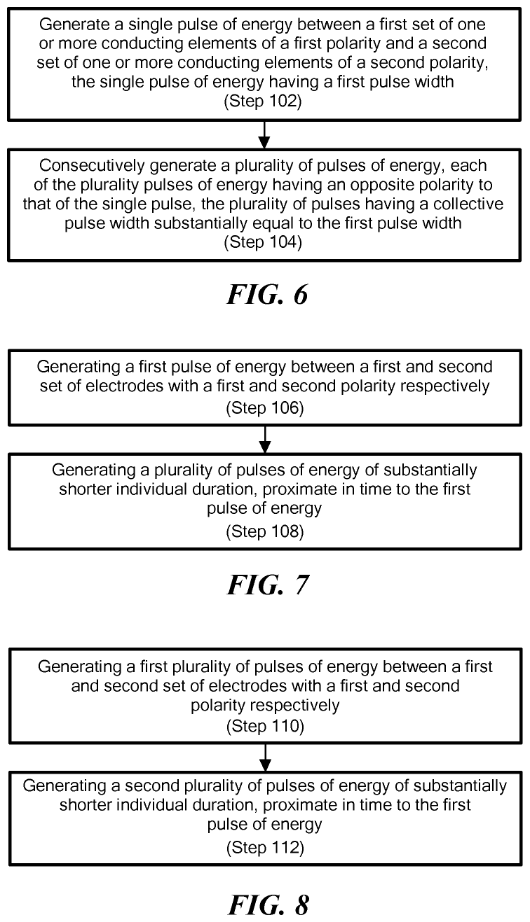

Referring now to FIG. 6, in an exemplary method of treating tissue with pulsed field ablation energy includes generating and delivering a single pulse of energy between a first set of one or more conducting elements, of a first polarity and a second set of one or more conducting elements of a second polarity, the single pulse of energy having a first pulse width (Step 102). For example, the generator 14 may be configured to generate a pulse having a first pulse width between two electrodes having opposite polarity. The single pulse may have a positive or negative voltage. Either before or after generating the single pulse, but consecutively, the generator further is configured to generate and deliver a plurality of pulses of energy, each of the plurality of pulses of energy having a opposite polarity to that of the single pulse and having a collective pulse width substantially equal to the first pulse width (Step 104). For example, if the single pulse is positive, the plurality of pulses is negative and collective have a pulse width substantially the same as the single pulse. The plurality of pulses may be any number of pulses that sum in width to that of the single pulse. It is considered that this application may reduce other risk factors such as temperature rise at the first or second delivery elements, for example, electrodes 26, 28. The presence of bubbles can cause rises in impedance associated with specific elements and their reduction may consequently reduce heating and associated risks such as blood embolization, charring, platelet aggregation, etc. The methods described here may be done similarly for those benefits. The method of treating tissues with pulsed field ablation energy may be performed by one or more of the processing circuitry 44, the processor 46, and the waveform unit 50.

Now referring to FIG. 7 is an exemplary method of applying therapeutic electric fields. The method includes generating a first pulse of energy between a first and second set of electrodes with a first and second polarity respectively; and generating a plurality of pulses of energy of substantially shorter individual duration, proximate in time to the first pulse of energy. In one or more embodiments, the plurality of pulses may have a collective pulse width substantially equal to the first pulse width. The first polarity may be anodic relative to the second polarity during the delivery of the first pulse and the first polarity may be cathodic relative to the second polarity during the delivery of the plurality of shorter pulses.

FIG. 8 depicts steps for a method of applying therapeutic electric fields. The method includes generating a first plurality of pulses of energy between a first and second set of electrodes with a first and second polarity respectively; and generating a second plurality of pulses of energy of substantially shorter individual duration, proximate in time to the first pulse of energy. In one or more embodiments, the second plurality of pulses have a collective pulse width substantially equal to the collective pulse width of the first plurality of pulses. The second plurality of pulses have a larger count of individual pulses than the first plurality of pulses. The first polarity may be anodic relative to the second polarity during the delivery of the first plurality of pulses and the first polarity may be cathodic relative to the second polarity during the delivery of the plurality of shorter pulses.

Note also here that while the term pulses are generally described and illustrated as idealized square waves, other waveforms such as sinusoidal pulses or any number of shapes are considered. The more generalized cumulative effect maintained in the desired waveform as a substantially similar charge-time integration between the opposing polarity configurations during the biphasic delivery. The matching of cumulative pulse widths being a special case as described here as an ideal implementation in the current invention.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of ablating tissue with pulse field ablation energy, comprising:
generating a single pulse of energy between a first set of one or more conducting elements of a first polarity and a second set of one or more conducting elements of a second polarity, the single pulse of energy having a first pulse width; and
consecutively generating a plurality of pulses of energy, each of the plurality of pulses of energy having a opposite polarity to that of the single pulse, the plurality of pulses having a collective pulse width substantially equal to the first pulse width.

2. The method of claim 1, wherein the single pulse has a voltage between 300V and 4000V and a pulse width of 2 and 1000 μs.

3. The method of claim 2, wherein the plurality of pulses has a voltage between 300V and 4000V and a pulse width of 0.5 to 100 μs.

4. The method of claim 1, wherein the tissue being ablated is cardiac tissue.

5. The method of claim 1, wherein the first polarity and the second polarity are continually switched during subsequent generations of the single pulse of energy and the plurality of pulses of energy.

6. The method of claim 1, wherein the generating of the single pulse of energy occurs between a first electrode of the first set of one more conducting elements and a second electrode of the second set of one or more conducting elements.

7. The method of claim 6, wherein the first set of one or more conducting elements is on a first medical device and the second set of one more conducting elements is on a second medical device different than the first medical device.

8. The method of claim 1, wherein the single pulse precedes the plurality of pulses of opposite polarity in delivery order.

9. A pulse field ablation energy generator, comprising:
a controller having processing circuitry being configured to:
generate a single pulse of energy between a first set of one or more conducting elements of a first polarity and a second set of one or more conducting elements of a second polarity, the single pulse of energy having a first pulse width; and
consecutively generate a plurality of pulses of energy with opposite polarity to that of the single pulse of energy, the plurality of pulses having a collective pulse width substantially equal to the first pulse width.

10. The generator of claim 9, wherein the single pulse has a voltage between 300V and 4000V.

11. The generator of claim 10, wherein the plurality of pulses has a voltage between 300V and 4000V.

12. The generator of claim 19, wherein the processing circuitry is further configured to continually switch the first polarity and the second polarity during subsequent deliveries of the single pulse of energy and the plurality of pulses of energy.

13. The generator of claim 10, wherein the processing circuitry is configured to be in communication with a medical device, and wherein the delivering of the single pulse of energy occurs from a first electrode of the first set of one or more conducting elements and a second electrode of the second set of one or more conducting element of the medical device.

14. The generator of claim 13, wherein the second electrode is larger than the first electrode.

15. The generator of claim 14, wherein the processing circuitry is configured to be in communication with a medical device, and wherein the generating of the plurality of pulses occurs between the first electrode and the second electrode of the medical device.

16. A medical system, comprising:
a pulse field ablation energy generator;
a controller in communication with the generator and including processing circuitry being configured to:
generate a single pulse of energy between a first set of one or more conducting elements of a first polarity and a second set of one or more conducting elements of a second polarity, the single pulse of energy having a first pulse width; and
consecutively generate a plurality of pulses of energy with opposite polarity, the plurality of pulses having a collective pulse width substantially equal to the first pulse width; and
one or more medical devices having a plurality of electrodes in communication with the generator, the medical device having a first tip electrode and a proximal second electrode, the first tip electrode and the proximal electrode being configured to deliver the single pulse of energy and to deliver the plurality of pulses of energy.

17. The system of claim 16, wherein the processing circuitry is further configured to continually switch the first polarity and the second polarity during subsequent deliveries of the single pulse of energy and the plurality of pulses of energy.

18. The system of claim 17, wherein both the single pulse of energy and the plurality of pulses of energy have a voltage of between 300V to 4000V.
